(19) United States
(12) Patent Application Publication
(10) Pub. No.: US 2003/0170321 A1
(43) Pub. Date: Sep. 11, 2003

(54) Bioavailability enhancing activity of Carum carvi extracts and fractions thereof

(75) Inventors: Ghulam Navi Oazi, Jammu and Kashmir (IN); Kasturi Lal Bedi, Jammu and Kashmir (IN); Rakesh Kamal Johri, Jammu and Kashmir (IN); Manoj Kumar Tikoo, Jammu and Kashmir (IN); Ashok Kumar Tikoo, Jammu and Kashmir (IN); Subhash Chander Sharma, Jammu and Kashmir (IN); Tasdaq Abdullah, Jammu and Kashmir (IN); Om Parkash Suri, Jammu and Kashmir (IN); Bishan Datt Gupta, Jammu and Kashmir (IN); Krishan Avtar Suri, Jammu and Kashmir (IN); Naresh Kumar Satti, Jammu and Kashmir (IN); Ravi Kant Khajuria, Jammu and Kashmir (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/200,080

(22) Filed: Jul. 19, 2002

Related U.S. Application Data
(60) Provisional application No. 60/306,917, filed on Jul. 20, 2001.

(51) Int. Cl. A61K 35/78

(52) U.S. Cl. 424/725; 424/756

(57) ABSTRACT

The invention relates to the isolation of an extract and/ or its fraction from the plant Carum carvi, its standardization with its intended use as drug bioavailability and/or bioefficacy enhancer for the drugs belonging to therapeutic categories such as but not limited to antimicrobial, antifungal, anti-viral, antitubercular, antileprosy, antiinflammatory/anti-arthritic, cardiovascular, antihistaminics, respiratory distress relieving drugs, immunosuppressants, anti-ulcers, nutraceuticals in compositions to be administered orally/parenterally, topically, inhalations (including nebulizers), rectally, vaginally in human beings and/or veterinary conditions.

### BACKGROUND OF THE INVENTION

### FIELD OF THE INVENTION

The present invention relates to the use of bioavailability and/or bioefficacy enhancers—also termed as bioenhancers or BE and methods of their preparation which include their isolation from a natural source and obtaining the final products in their chemically characterized or fingerprint—profiled form.

The present invention is directed to preparation of active extracts/fraction from the plant Carum carvi which include their chemical characterisation, fingerprint profiling and methods of using such products to enhance bioavailability and/or bioefficacy of drugs, natural products and essential nutraceuticals. The present invention is directed to preparation of composite bioenhancers comprising polar and non-polar extracts of parts of Zingiber officinale and/or piperine (Ex: Piper nigrum and Piper longum) which increased significantly (50-180%) , the bioavailability of a number of classes of drugs, for example, but not limited to, antibiotics, antifungals, anti-virals, anticancer, cardiovascular, CNS, anti-inflammatory/anti-arthritic, anti-TB/antileprosy, anti-histaminic/respiratory dosorders, corticosteroids, immunosppressants, anti-ulcer. Such extracts/fractions of Carum carvi either in presence or absence of Zingiber officinale and/or piperine (Ex: Piper nigrum and Piper longum) have been found to be highly selective in their bioavailability/bioefficacy enhancing action.

There is a great interest and medical need for the improvement of bioavailability of a large number of drugs which are (a) poorly bioavailable, (b) given for long periods, and are (c) toxic and expensive. Maximizing oral bioavailability is therapeutically important because the extent of bioavailability directly influences plasma concentrations and consequently therapeutic efficacy and dose related toxic effects resulting after oral drug administration. Poorly bioavailable drugs remain sub-therapeutic because a major portion of a dose never reaches the plasma or exerts its pharmacological effect unless and until very large doses are given which may lead to serious side effects. Any significant improvement in bioavailability will result in lowering the dose or the dose frequency of that particular drug. Besides, inter-subject variability is inversely correlated with the extent of bioavailability. Therefore, low oral bioavailability leads to high variability and poor control of plasma concentration and pharmacodynamic effects. Inter-subject variability is particularly of concern for a drug with a narrow safety margin.

Incomplete oral bioavailability has various causes. These include poor dissolution or low aqueous solubility, poor intestinal membrane permeation, degradation of the drug in gastric or intestinal fluids and pre-systemic intestinal or hepatic metabolism. The normal practice to offset some of these problems has been to increase the dosage as stated earlier which has the concerns of toxicity patients' non-compliance.

Many therapeutic treatments are also accompanied by loss of essential nutraceuticals in the course of therapy. The present invention improves nutritional status by increasing bioavailability/bioefficacy of various nutraceuticals also which include metals and vitamins. The bioenhancers of the invention also have the potential to enhance the bioefficacy of a drug without influencing its plasma concentrations for various reasons, some of which, but not limited to, are described later in this invention under Section on ‘Bioavailability/Bioenhancing activity’.

### DESCRIPTION OF RELATED ART

Several approaches have been adopted in the past to maximize oral bioavailability, such as (a) particle size reduction (micronization, nanonization, etc.,) (b) polymorphic or crystal size and form selection, (c) solubilization of lesser soluble drugs by way of chemical modifications, complexation and use of co-solvents/surfactants, (d) targeted delivery of drug at the site of action, (e) controlled drug delivery by film coating or use of polymeric matrices for sustained release of drugs, (f) prodrug approach, and (g) microencapsulation using liposomes.

However, based on clues from Ayurvedic literature, a new approach of increasing the bioavailability of drugs including poorly bioavailable drugs had been conceptualized at RRL, Jammu. One of the groups of herbals which has been documented very frequently as essential part of about 70% of Ayurvedic prescriptions, was noted to be ‘Trikatu’, that comprises three acrids viz. long pepper, black pepper and dry ginger in equal proportions. A single major alkaloidal constituent from peppers (piperine) was found to be responsible for bioavailability enhancing effect. The role of ginger is to regulate intestinal function to to facilitate absorption. Influence of piperine was extensively studied on anti-TB drugs. It was determined that in combination with piperine the dose of rifampicin can be reduced by about 50% while retaining the therapeutic efficacy of this anti-TB drug at par with the standard dose (450 mg). Based on these findings several other reputed plants were evaluated for bioavailability/bioefficacy enhancing activity. Polar and non-polar extracts of parts of a few plants viz., Zingiber officinale, and Cuminum cyminum increased significantly (25-300%), the bioavailability of a number of classes of drugs, for example, but not limited to, antibiotics, antifungals, anti-virals, anticancer, cardiovascular, CNS, anti-inflammatory/anti-arthritic, anti-TB/antileprosy, anti-histaminic/respiratory disorders, corticosteroids, immunosuppressants, anti-ulcer. Such extracts either in presence or absence of piperine have been found to be highly selective in their bioavailability/bioefficacy enhancing action.

### DESCRIPTION OF THE PREFERRED EMBODIMENTS

Carum carvi

It is one of the most prized culinary herb which is used extensively in India. The herb finds very frequent mention in Ayurved and other Indian Systems of Medicine prescriptions used against a wide variety of ailments. It remained a scientific curiosity that a single plant can have biological activities for such a large variety of ailments or diseases for which these prescriptions are employed.

Chemistry of Carum carvi

Carum carvi Linn seeds are known as Jira (Beng.), Shahjiru (Guj.), Kala jira, Shiajira (Hindi), Shalajira (Mar.) Carum carvi is an annual or biennial glabrous herb, 30-100 cm in height, native to Europe and West Asia, found growing wild in Himachal Pradesh and cultivated in the hills and plains of North India and in the hills of South India for its aromatic seeds.

Its seeds are widely used as a spice for culinary purposes and for flavouring bread, biscuits, cakes, candies, cheese, curries, pickles, sausages, meat products, confectionery and liqueurs of kummel type. They are also used as a flavouring constituent in cordials and in certain preparations of Cannabis. In medicine, they are used as carminative, mild stomachic, aromatic and diuretic. Both the seeds and the essential oils (caraway oil) are prescribed in flatulent colic and stomach derangements. In patients suffering from lumbago and rheumatism, exposing the affected parts to the vapours from the seeds gives relief from the disease. The alcoholic extract of the fruits show dose-dependent antispasmodic effect. Its water finds use as a vehicle for paediatric medicines. Hexane extract of the fruits was found to have excellent larvicidal activity against the mosquito Culex pipiens fatigans Wiedm. [Marketing of Minor Spices in India, 1968, 119; Krishna & Badhwar, J. Sci. Industr. Res., 1952, 11A, suppl., 259; Sharma and Kapil, Loc. cit.; Embong et al Canad J Pl Sci., 1977, 57, 543; I.P., 1966, 104; I PC., 55; Gharat, Pharmaceutist, 1958-59, 4 (2), 21; Cappelletti et al., J. Ethnopharmacol, 1982, 6, 178; Forster et al, Planta Med, 1980, 40, 309; Deshmukh et al, Pesticides, 1982, 16 (12), 7]. The dried crushed seeds, on steam distillation, gave a pale yellow to light brown essential oil (known as caraway oil) with a strong aromatic odour. The oil content of the seeds varies according to the degree of maturity of the seeds. Storage affects the oil content of seeds up to 2.8 per cent per annum. All Indian samples of the seeds contained 5.8-8.1 per cent of caraway oil. Carvone and the limonene are the chief constituents of the oils and its odour and flavour are mainly attributed to them. Other constituents present in the oil are α- and β-pinene and ρ-cymene. Besides the above constituents, camphene, Δ³-carene, dihydrocarvone, β-fenchene, myrcene, α- and β-phellandrene, sabinene, α and γ-terpinene, α-thujene, terpinolene, tricyclene, d- and 1-dihydropinol, 1-neodihydrocarveol, 1-isodihydrocarveol, carveol, d-dihydrocarveol, acetaldehyde, methyl alcohol, furfural have also been isolated from European caraway oil (Arctander, 124; Dijkstra and Speckmann, loc. cit.; Atal & Sood, Indian J Pharm, 1967, 29, 42; I.P., 1966, 105; Padha et al Parfum u Kosmetik, 1969, 50, 296; Chem Abstr. 1980, 93, 191892; Salveson & Svendsen, Planta Med. 1976, 30, 93, Guenther, IV, 582).

Caraway oil is primarily used like caraway seeds in flavouring several food products, and in medicine as carminative. It is the main ingredient in the scandinavian “snaps” and the German “kummel”. It is employed in gargle preparations, toothpaste flavours, chewing gum, candy and as a masking agent in bad tasting pharmaceutical preparations and obnoxius insecticides. It also exhibits neurotropic anti-spasmodic activity. In mixture with alcohol and castor oil, it is used for the treatment of scabies. The essential oil shows moderate anti-bacterial and anti-fungal property against several bacteria and fungi. Decarvonised oil is sold in the market for scenting cheap soaps, in jasmine bases and tabac perfumes (IPC., 54; Arctander, 125; Chopra et al, 1958, 92; Chem Abstr, 1968, 68, 48218; Narayan et al Indian Drugs, 1979-80, 17, 394; El-keltawi et al, Herba pol, 1980, 26, 245).

The seeds also contain 3-glucosides and 3-galactosides of kaempferol, quercetin and isorhamnetin, and a hydrocarbon (m p, 62-63°). Presence of 5-methoxy-, and 8-methoxy psoralens, sterol, umbelligerone, scopoletin and herniarin is also reported. The fatty acid composition of the oil is: palmitic, 3.6; oleic, 60.7; linoleic, 19.6 and petroselinic, 17.0% (Food technol Abstr., 1974, No. 93, 470; Harborne & Williams, Phytochemistry, 1972, 11, 1741; Ceska et al., ibid, 1987, 26, 165; Chakraborti, Trans Bose Res Inst, 1956-58, 21, 61; Chem. Abstr., 1969, 71, 57561; Hilditch & Williams, 287).

Bioavailability/Bioefficacy Enhancing Activity

The aqueous, aqueous—alcoholic, ketonic, ethereal, halogenated solvents extracts of the plant parts were evaluated with different therapeutic categories of drugs and nutraceutical (vital amino acids, metals, antioxidants, vitamins) and herbal drugs. The bioavailability/bioefficacy enhancing (BE) activity of Carum carvi extracts was found to be consistent from 5 mg to 100 mg irrespective of the amount of the drug(s) present in the formulation. Sub-fractions of the active extracts were also evaluated, with the same categories of drugs. The doses of the fraction(s) responsible for the BE activity ranged from 1.0 to 55 mg. The parent extract as well as the active fraction(s) were found to be active individually as well as in combination with each other with different categories of drugs.

The individual extract or its fractions were found to be 20-110% more active when used in combination with bioenhancer products developed from Zingiber officinale. The effective range for Zingiber officinale. BEs was 10-150 mg. Besides both the parent extracts as well as their fractions from Carum carvi in different combinations showed pronounced activity ranging from 25-95% in presence of piperine. The amount of piperine in these formulations ranged from 3-15 mg. The extracts or its fractions either in presence or absence of BEs from Zingiber officinale. and/or piperine have been found to be highly selective in their bioavailability and/ or bioefficacy enhancing activity. This is apparent from the degree of bioavailibility and/or bioefficacy enhancement caused by these extracts/fractions as exmeplified in accompanying examples. The reasons for this selective pattern may be attributable to one or more than one of the following reasons: (a) Promoting the absorption of drugs from GIT, (b) Inhibiting or reducing the rate of biotransformation of drugs in the liver or intestines, (c) Modifying the immune system in a way that the overall requirement of the drug is reduced substantially, (d) Increasing the penetration or the entry into the pathogens even where they become persistors within the macrophages such as for Mycobacterium tuberculosis and such others. This eventually ensures the enhanced killing of these organisms well secured within the places otherwise inaccessible to the active drug, (e) Inhibiting the capability of pathogens or abnormal tissue to reject the drug e.g., efflux mechanisms frequently encountered with anti-malarial, anti-cancer and anti-microbial drugs, (f) Modifying the signalling process between host and pathogen ensuring increased accessibility of the drugs to the pathogens, (g) Enhancing the binding of the drug with the receptors like proteins, DNA, RNA, etc., in the pathogen, thus potentiating and prolonging its effect leading to enhanced antibiotic activity against pathogens, (h) Besides above plausible modes of action, the bioenhancer agents may also be useful for promoting the transport of nutrients and the drugs across the blood brain barrier, which could be of immense help in the control of diseases like cerebral infections, epilepsy and other CNS problems.

Primarily, but not exclusively, the invention enhances the carrier mediated entry of drugs and also the passive diffusion and the active transport pathways in the tissue which are responsible for transporting physiological substances such as nutraceuticals to their target sites. As applicable to any mechanism of action the products of this invention contribute in a synergistic and/or additive manner so that most drugs and nutraceuticals in presence of the products described in the present art are more bioavailable or bioefficaceous as a result of one or more of these mechanisms. The bioavailability and/or bioefficacy of drugs and nutraceuticals is also relevent to animal health besides being important for humans. The invention therefore is also intended to be used in veterinary preparations.

### EXAMPLES

The following examples are intended to demonstrate some of the preferred embodiments and in no way should be construed so as to limit the scope of the invention. Any person skilled in the art can design more formulations, which may be considered as part of the present invention.

Example 1 Preparation of colourless, non-pungent piperine by a novel process as already claimed (Indian Patent No 1726890)

Example 2. Preparation of fully characterized BEs from Zingiber officinale as already claimed in a copending patent.

Example 3. List of drugs cited below as some of the examples for the purpose of the present invention.

| | Categories | Drugs |
|---|---|---|
| I. | Antibiotics | Fluoroquinolones: Cipro-, nor-, P-, and O-floxacins |
| | | Macrolides: Erythro-, roxythro-, and Azithromycin |
| | | Cephalosporins: Cefixime, Cefalexin, Cefadroxil and |
| | | Cefatrioxone, cefidinir |
| | | Penicillins: Amoxycillin Cloxacillin |
| | | Aminoglycosides: Amikacin, Kanamycin |
| II. | Antifungal | Fluconazole, Amphotericin B, Ketoconazole |
| III. | Anti-viral | Acyclovir, Zidovudine |
| IV. | Anti-cancer | Methotrexate, 5-Fluorouracil, Dauxorubicin, Cisplatin, |
| | | Adriamycin |
| V. | CVS drugs | Amlodipine, Lisinopril, Atenolol |
| VI. | CNS drugs | Alprazolam Haloperidol |
| VII. | Anti-inflammatory | Diclofenac, Piroxicam, Nimesulide, Rofecoxib |
| | Antiarthritic | |
| VIII. | Anti-TB/Antileproxy | Rifampicin, Isoniazid, Pyrazinamide, Ethambutol, Dapsone |
| | drugs | |
| IX. | Anti histamines/ | Salbutamol, Theophylline, Bromhexine, Loretidine |
| | respiratory disorders | |
| X. | Carticosteroids | Prednisolone, dexamethsone, betamethasone |
| XI. | Immunosuppressants | Cyclosporins, Tacrolimus, Mycophenolate mofetil |
| XII. | Anti-ulcer | Ranitidine, Cimetidine, Omeprazole |

### EXAMPLE 3

(i): Antibiotics:

(a) Fluroquinolones

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Ciprofloxacin | 78 | 55 | 110 | 68 | 133 |
| P-floxacin | Nil | 61 | 70 | 53 | 75 |
| O-floxacin | 65 | 52 | 167 | 49 | 170 |
| Norfloxacin | 55 | nil | 65 | nil | 60 |

(b) Macrolides

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Erythromycin | 70 | 95 | 100 | 68 | 105 |
| Roxythromycin | 65 | 110 | 95 | 72 | 98 |
| Azithromycin | 55 | 89 | 90 | 78 | 86 |

(c) Cephalosporins

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Cefalexin | Nil | 90 | 90 | 75 | 79 |
| Cefadroxil | 67 | 70 | 95 | 68 | 85 |
| Cefatrioxone | 72 | Nil | 78 | Nil | 75 |
| Cefixime | 80 | nil | 79 | nil | 82 |
| Cefidinir | 89 | 60 | 95 | 35 | 130 |

(d) Penicillins

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Amoxycillin | 75 | 120 | 115 | 80 | 100 |
| Cloxacillin | 110 | 87 | 95 | 76 | 110 |

(e) Aminoglycosides

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Amikacin | 85 | nil | 100 | nil | 92 |
| Kanamycin | Nil | 72 | 87 | 65 | 68 |

(ii) Antifungal

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Fluconazole | 65 | 87 | 98 | 120 | 110 |
| Amphotericin | 78 | nil | 90 | nil | 80 |
| B | | | | | |
| Ketoconazole | 55 | 105 | 100 | 125 | 96 |

(iii) Anti-Cancer

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Methotrexate | 76 | 65 | 89 | 87 | 102 |
| 5-Fluorouracil | 90 | 87 | 110 | 110 | 100 |
| Dauxorubicin | Nil | 68 | 70 | 72 | 69 |
| Cisplatin | Nil | nil | nil | 56 | 55 |

(iv) Cardiovascular

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Amlodipine | Nil | 43 | 50 | 68 | 65 |
| Lisinopril | 79 | 85 | 95 | 76 | 90 |
| Atenolol | 100 | nil | 93 | nil | 97 |
| Propranolol | 68 | 84 | 90 | 76 | 75 |

(v) Anti-Viral

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Acyclovir | 78 | 96 | 100 | 82 | 90 |
| Zidovudine | 92 | 140 | 95 | 105 | 87 |

(vi) CNS Drugs:

| | % Enhancement in bioavailability | | | | |
|---|---|---|---|---|---|
| | BE from | | Carun | BE from | Carum carvi |
| | Carum | Piperine | carvi + | Zingiber | + Zingiber |
| | carvi | as BE | Piperine | officinale | officinale |
| Alprazolam | Nil | 65 | 70 | 76 | 80 |
| Haloperidol | 95 | nil | 90 | nil | 85 |

(vii) Anti-Inflammatory/Antiarthritic:

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Diclofenac | Nil | 120 | 100 | 90 | 95 |
| Piroxicam | Nil | 110 | 98 | 86 | 76 |
| Nimesulide | 100 | 132 | 140 | 144 | 145 |
| Rofecoxib | 75 | nil | 70 | nil | 80 |

(viii) Anti-TB/Antileprosy Drugs:

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Rifampicin | 110 | 45 | 170 | 65 | 140 |
| Isoniazid | Nil | nil | nil | nil | nil |
| Pyrazinamide | 45 | nil | 50 | nil | 55 |
| Ethambutol | Nil | nil | nil | nil | nil |
| Dapsone | 56 | 34 | 67 | 46 | 68 |
| Ethionamide | 68 | 45 | 65 | 56 | 70 |
| Cycloserine | 70 | 67 | 80 | 71 | 75 |

(ix). Anti-Histamines/Respiratory Disorders:

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Salbutamol | 75 | 60 | 89 | 78 | 80 |
| Theophylline | 70 | 65 | 79 | 76 | 89 |
| Bromhexine | Nil | 67 | 70 | 67 | 71 |
| Loratidine | 76 | nil | 70 | nil | 80 |

(X) Corticosteroids:

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Prednisolone | 65 | nil | 67 | nil | 60 |
| Dexamethasone | 72 | 66 | 77 | 76 | 73 |
| Betamethasone | 80 | 72 | 89 | 75 | 77 |

(xi) Immunosuppressants:

| | % Enhancement in bioavaiiability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Cyclosporin A | 100 | nil | 105 | 116 | 120 |
| Tacrolimus | 90 | 105 | 95 | 75 | 114 |
| Mycophenolate | Nil | nil | nil | nil | nil |
| Mofeit | | | | | |

(xii) Anti-Ulcer

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Ranitidine | 67 | 21 | 70 | 147 | 150 |
| Cimetidine | 72 | nil | 84 | 98 | 100 |
| Omeprazole | 76 | nil | 70 | nil | 75 |

D. Herbal Formulations

| | % Enhancement in bioavailability | | | | |
|---|---|---|---|---|---|
| | BE from | | Carum | BE from | Carum carvi |
| | Carum | Piperine | carvi + | Zingiber | + Zingiber |
| | carvi | as BE | Piperine | officinale | officinale |
| Echinacea | Nil | 75 | 76 | 66 | 65 |
| augustifolia | | | | | |
| Tinospora | 76 | 85 | 90 | 67 | 71 |
| cordifolia | | | | | |
| Picrorrhiza | 80 | 78 | 110 | 56 | 76 |
| kurroa | | | | | |
| Aegles | 65 | Nil | 65 | Nil | 60 |
| marmelos | | | | | |
| Andrographis | 68 | 63 | 72 | 55 | 54 |
| paniculata | | | | | |
| Emblica ribes | Nil | Nil | nil | 65 | 68 |
| Asparagus | Nil | 58 | 55 | 44 | 45 |
| racemosus | | | | | |
| Terminalia | 92 | Nil | 87 | Nil | 91 |
| chebula | | | | | |
| Withania | 76 | 55 | 70 | 64 | 76 |
| somnifera | | | | | |
| Centella | 68 | nil | 65 | nil | 62 |
| asiatica | | | | | |

B. Nutraceutical

| | Category |
|---|---|
| I. | Vitamins |
| | Vitamin A |
| | Vitamin E |
| | Vit. B1 |
| | Vit. B6 |
| | Vit. B12 |
| | Vit. C |
| | Folic acid |
| II. | Antioxidants |
| | β-Carotene |
| | Silymarin |
| | Selenium |
| III. | Natural herbal products |
| | Curcumin |
| | Boswellic acid |
| | Rutin |
| | Piperine |
| IV. | Essential nutritional components |
| | Methionine |
| | Lysine |
| | Leucine |
| | Valine |
| | Isolencine |
| | Zinc |
| | Calcium |
| | Glucose |
| | Potassium |
| | Copper |
| | Iron |

1. The invention relates to the isolation of an extract and/or its fraction from the plant Carum carvi, its standardization with its intended use as drug bioavailability and/or bioefficacy enhancer for the drugs belonging to therapeutic categories such as but not limited to antimicrobial, antifungal, anti-viral, antitubercular, antileprosy, antiinflammatory/anti-arthritic, cardiovascular, antihistaminics, respiratory distress relieving drugs, immunosuppressants, anti-ulcers, nutraceuticals in compositions to be administered orally/parenterally, topically, inhalations (including nebulizers), rectally, vaginally in human beings and/or veterinary conditions.

2. The invention relates to the preparation of a formulation containing extract and/or its fraction/from the plant Carum carvi and piperine, its standardization with its intended use as drug bioavailability and/or bioefficacy enhancer for the drugs belonging to therapeutic categories such as antimicrobial, antifungal, anti-viral, antitubercular, antileprosy, antiinflammatory, antiarthritic, cardiovascular, antihistaminics, respiratory distress relieving drugs, immunosuppressants, anti-ulcers, nutraceuticals in compositions to be administered orally/parenterally, topically, inhalations (including nebulizers), rectally, vaginally in human beings and/or veterinary conditions.

3. The invention relates to the preparation of a formulation containing extract and/or its fraction from the plant Carum carvi and Zingiber officinale, its standardization with their intended use as drug bioavailability and/or bioefficacy enhancer for the drugs belonging to therapeutic categories such as antimicrobial, antifungal, anti-viral, antitubercular, antileprosy, antiinflammatory, antiarthritic, cardiovascular, antihistaminics, respiratory distress relieving drugs, immunosuppressants, anti-ulcers, nutraceuticals in compositions to be administered orally/parenterally, topically, inhalations (including nebulizers), rectally, vaginally in human beings and/or veterinary conditions.

4. The bioavailability/bioefficacy enhancer principle may be any extract, its fraction or pure molecule isolated from the plant.

5. Any drug may be selected from the therapeutic categories such as those mentioned in claim 1.

6. A process for the preparation of piperine from Piper nigrum, Piper longum, or its oleoresin.

7. A process for the preparation of extract(s)/fraction(s) from Zingiber officinale

8. A process for the preparation of extract(s)/fraction(s) of plants which may involve the use of water, alcohol, combinations of water and alcohol, halogenated hydrocarbons, ketones, ethers as solvents.

9. A process for the preparation of extract(s) having piperine which may involve the use of water, alcohol, combinations of water and alcohol, halogenated hydrocarbons, ketones, ethers as solvents.

10. A process for preparation of composite bioenhancers having active extracts/fractions from Carum carvi and/or Zingiber officinale with or without piperine making use of physical techniques like dialysis/molecular sieves/membranes, variety of chromatographic techniques and/or liquid-liquid or solid phase extractions, followed by their complete finger print profiles (HPLC/HPTLC/LC-MS-MS)

11. The combination/s of bioenhancre/s having active extract/fraction do not represent a mere physical mixing but a specialized process for the purpose of formulations that may involve chemical techniques like particle size reduction, use of selective polar solvents or use of ionic/non-ionic surfactants.

12. The formulation of a drug selected from any of the therapeutic categories of the drugs, nutraceuticals, herbal drugs/formulations in combination with the bioenhancer may be intended for routes of administration viz., oral, parenteral, nasal, inhalation including nebulisers, rectal, vaginal, transdermal and others.

13. The bioenhancing effect of the extracts/fractions of Carum carvi either alone or in combination with extracts/fractions of Zingiber officinale and/or piperine is selective, as shown but not limited to the accompanying examples and does not enhance the bioavailability/bioefficacy of each and every drug, nutraceutical, herbal drug/formulation.

14. That the plant extracts/fractions either individually or in combination express no biological or toxicological effect of their own at the doses at which they are intended to be used.
